(19) United States
(12) Patent Application Publication
(10) Pub. No.: US 2016/0114193 A1
(43) Pub. Date: Apr. 28, 2016

(54) MULTILAYER ULTRASOUND TRANSDUCERS FOR HIGH-POWER TRANSMISSION

(71) Applicant: Oleg Prus, Haifa (IL)

(72) Inventor: Oleg Prus, Haifa (IL)

(21) Appl. No.: 14/521,734

(22) Filed: Oct. 23, 2014

(51) Int. Cl. A61N 7/00; G06F 17/50; G10K 11/18

(52) U.S. Cl. CPC A61N 7/00; G10K 11/18; G06F 17/5009

(57) ABSTRACT

A multilayer ultrasound transducer is used to provide high output power with a desired transmission and reception frequency response profile.

### FIELD OF THE INVENTION

The present invention relates, generally, to ultrasound systems. In particular, various embodiments are directed to multilayer ultrasound transducers for high-intensity transmission.

### BACKGROUND

Focused ultrasound (i.e., acoustic waves having a frequency greater than about 20 kilohertz) can be used to image or therapeutically treat internal body tissues within a patient. For example, ultrasonic waves may be used to ablate tumors, eliminating the need for the patient to undergo invasive surgery. For this purpose, a single-plate, piezo-ceramic transducer may be placed externally to the patient, but in close proximity to the tissue to be ablated (“the target”). The transducer converts an electronic drive signal into mechanical vibrations, resulting in the emission of acoustic waves. The transducer may be shaped so that the waves converge in a focal zone. Typically, the transducer functions in a vibrational mode along the acoustic emission direction and has a high aspect ratio of the lateral dimensions (i.e., length or width) to the thickness. Single-plate transducers tend to have power-delivery efficiencies of 50%-60% and a bandwidth of approximately 10% of the center frequency. Single-transducer designs have advantages such as low cost and possibility of effective power transmission (e.g., at odd harmonics of the resonant frequencies) but suffer from low focal-zone steering angles and limited frequency range.

Alternatively, the transducer may be formed of a two-dimensional grid of uniformly shaped piezoelectric transducer elements (or “rods”) glued, via a polymer matrix, to a matching conductive substrate. Typically, each transducer element transmits acoustic waves along the direction of rod elongation and can be driven individually or in groups; thus the phases of the transducer elements can be controlled independently from one another. Such a “phased-array” transducer facilitates focusing the transmitted energy into a focal zone and steering the focal zone to different locations by adjusting the relative phases between the transducer elements and/or simultaneously generating multiple foci to treat multiple target sites by grouping the transducer elements. Although phase-array transducers tend to have bandwidths of 30%-40%, they are less capable of high-power transmission (compared with the single-plate transducer) due to poor thermal stability and low thermal conductivity of the polymer matrix. In addition, because the intensity at the third harmonic of the transducer resonant frequencies may be damped by the polymer matrix, the phase-array transducer typically cannot transmit sufficient power at a frequency above the base harmonic. The working frequency may be adjusted to a frequency lower than the resonant frequencies—in particular, during ultrasound imaging or sensing (e.g., using hydrophones)—but high-power transmission in this frequency regime is challenging due, for example, to create an impedance mismatch between the driving circuitry and the transducer.

Accordingly, there is a need for ultrasound transducers that efficiently deliver high power output at desired multiple frequency bands.

### SUMMARY

The present invention provides, in various embodiments, an ultrasound transducer that can deliver a high-power output with a desired transmission and reception frequency response profile. In one implementation, the transducer includes a multilayer structure laminated in a stacked configuration between two electrode layers for providing a high-power delivery efficiency; the number and order of the multiple layers and the material and thickness of each layer may be selected based on the desired frequency-response profile. Because each layer may have a different acoustic parameters, a combination of layers can generate a desired working frequency response with suitable bandwidth. In various embodiments, at least one of the layers is formed of a piezoelectric material that can be driven by electric signals to produce ultrasound energy; all other layers should efficiently deliver the electrical energy. To ensure this, they should have at least one of the following properties:
a. Non-zero isotropic electrical conductivity or volume resistivity typically less than 5MΩ×m/F, where F is a typical working frequency (in Hz).
b. Non-zero, anisotropic (in the z-direction, i.e., along the acoustic axis) electrical conductivity or volume resistivity typically less than 5MΩ×m/F, where is a typical working frequency (in Hz). These properties may be provided, for example, by one or more conductive vias.
c. High capacitance to ensure that the volume electrical impedance is below a threshold, e.g., 5MΩ×m/F where F is a typical working frequency (in Hz).

The entire stack of the multilayer transducer then functions as a single-plate transducer or a composite phase-array transducer having multiple transducer elements that are formed by segmenting the transducer stack. In one embodiment, no functional layers are required outside the two electrode layers of the transducer. As used herein, the term “functional layers” refers to layers that contribute to ultrasound energy transmission and reception. Thus, the current invention provides an approach to design multilayer transducers in accordance with the acoustic and electromechanical properties of the materials of each layer for achieving a high-power output with a desired frequency response profile.

Accordingly, in one aspect, the invention pertains to a transducer for delivering acoustic energy to a target site within a patient. In various embodiments, the transducer includes one or more piezoelectric layers, multiple electrically conductive layers, and two electrode layers. In one implementation, the piezoelectric layer(s) and the electrically conductive layers are positioned between the two electrode layers to form a stacked configuration that provides a desired power output and transmission and reception frequency responses. The piezoelectric layer(s), for example, may include ceramic, single crystal, polymer and co-polymer material, and/or ceramic-polymer. The electrically conductive layers may have a volume resistivity of less than 5 MΩ×m/F, where F denotes the working frequency of the transducer. The electrically conductive layers, for example, may include metal, graphite, carbon, plastic, and/or conductive fiber composite.

In various embodiments, the transducer further includes one or more interlayers connecting the piezoelectric layer(s) and the electrically conductive layers. The interlayer(s) may each include, consist of or consist essentially of metal, graphite, carbon, metal-coated polymer, glass, and/or ceramic for ensuring conductivity and lamination between the piezoelectric layer(s) and the electrically conductive layers. Additionally, the transducer may include a dielectric layer stacked between the two electrode layers; the dielectric layer may include a ceramic or a depoled piezo-ceramic. In some embodiments, the transducer includes an impedance-matching layer having a predetermined acoustic and/or electrical impedance and thickness. In one implementation, the transducer includes no functional layers outside the two electrode layers.

In another aspect, the invention relates to a method of manufacturing and using a transducer. In various embodiments, the method includes providing a single piezoelectric layer and multiple electrically conductive layers; laminating the single piezoelectric layer and the electrically conductive layers in a stacked configuration; applying one electrode layer on top of the stack and one electrode layer on bottom of the stack to form a transducer; and applying a voltage to the transducer for causing the transducer to emit acoustic energy. Critically, the material, thickness, and/or order of the layers is determined based on a desired power output and transmission and reception frequency responses.

The method may further include providing multiple interlayers connecting the single piezoelectric layer and the electrically conductive layers. Additionally, the method may include providing a dielectric layer stacked between the two electrode layers. In some embodiments, the method includes providing an impedance-matching layer having a predetermined acoustic and/or electrical impedance and thickness; the acoustic and/or electrical impedance of the impedance-matching layer is determined based on acoustic and/or electrical properties of the transducer. Further, the method may include segmenting the transducer into multiple elements (e.g., using laser cutting or dicing) for creating a composite phase-array transducer. In one embodiment, the electrode layers are added to the stack using evaporation or sputtering that provides a conformal coating to surfaces of the stack.

Still another aspect of the invention relates to a method of designing and manufacturing a transducer based on a desired power output and transmission and reception frequency responses. In various embodiments, the method includes computationally simulating behavior of one or more piezoelectric layers and one or more electrical conductive layers; adjusting (a) a number of layers, (b) an order of layers, and/or (c) a thickness of the layers until the computationally simulated behavior conforms to the desired power output and transmission and reception frequency responses; and producing the computationally simulated transducer by: providing one or more piezoelectric layers and one or more electrical conductive layers corresponding to the computationally simulated layers; laminating the piezoelectric layer(s) and the electrical conductive layer(s) in a stacked configuration; and applying one electrode layer on top of the stack and one electrode layer on bottom of the stack.

In another aspect, the invention relates to a system for delivering acoustic energy to a target site within a patient. In various embodiments, the system includes a transducer having multiple layers including one or more piezoelectric layers, multiple electrically conductive layers, and two electrode layers configured in a stacked configuration; driver circuitry for providing electrically drive signals to the transducer; and a controller coupled to the driver circuitry for controlling the drive signals.

As used herein, the terms “approximately” and “substantially” mean±10%, and in some embodiments, ±5%. The term “consists essentially of” means excluding other materials that contribute to function, unless otherwise defined herein. Nonetheless, such other materials may be present, collectively or individually, in trace amounts. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

The foregoing will be more readily understood from the following detailed description of the invention in conjunction with the drawings, wherein:

FIG. 1A and 1B schematically depict exemplary focused ultrasound systems having a single-plate transducer and a composite phase-array transducer, respectively, in accordance with various embodiments of the present invention;

FIGS. 2A and 2B are schematic cross-sectional views of multilayer ultrasound transducers in accordance with various embodiments of the present invention;

FIG. 3 shows a plot of the simulated and measured admittance spectrum of the ultrasound transducer in accordance with various embodiments of the present invention;

FIG. 4 depicts simulated and measured power-delivery efficiency of the ultrasound transducer as a function of frequencies in the range of 200-300 kHz in accordance with various embodiments of the present invention;

FIG. 5 depicts simulated and measured power-delivery efficiency of the ultrasound transducer as a function of frequencies in the range of 500 kHz to 1 MHz in accordance with various embodiments of the present invention; and

FIG. 6 is a flow chart illustrating a method of designing, manufacturing, and using the multilayer transducer in accordance with various embodiments of the present invention.

### DETAILED DESCRIPTION

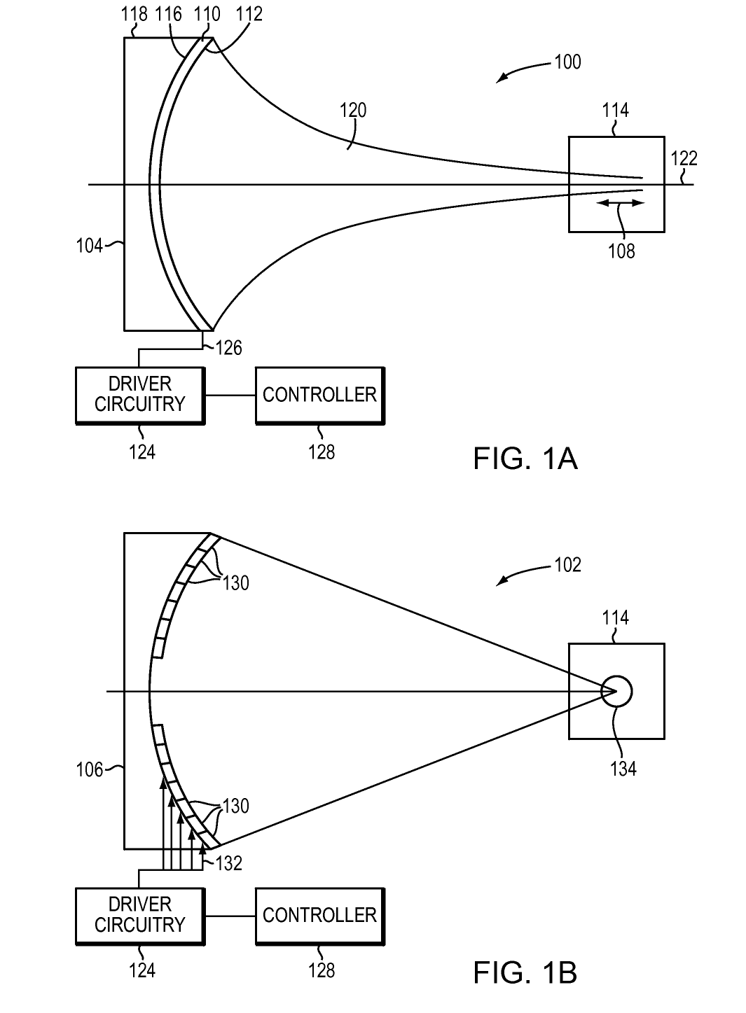

FIGS. 1A and 1B depict exemplary focused ultrasound systems 100, 102 having a single-plate transducer 104 and a composite phase-array transducer 106, respectively, in accordance with embodiments of the present invention, although alternative systems with similar functionality are also envisioned. As shown, a single-plate ultrasound transducer 104 may have a spherical concave surface in three dimensions (i.e., resembling a bowl); the curved transducer 104 may focus acoustic energy over a target region 108. Typically, the transducer 104 comprises a curved, piezoelectric element 110 having one electrode 112 on the front side, facing subject 114, and one electrode 116 on the opposite, rear side, facing away from the subject 114. In some embodiments, the transducer 104 includes a matching layer 118 to match the acoustic and/or electrical impedance of the transducer 104 to that of transducer supporting circuitry. The coupling medium 120 in contact with both the transducer 104 and the subject 114 may be water or a gel having a density similar to that of water. The focal region 108 is a relatively small and concentrated region around an axis 122 passing through the geometric center of spherical transducer 104. The ultrasound system 100 includes driver circuitry 124 for providing electrical drive signals 126 to the transducer 104, and a controller 128 for controlling the drive signals 126 provided by the driver circuitry 124. The controller 128 may be implemented in hardware, software or a combination of the two. For embodiments in which the functions are provided as one or more software programs, the programs may be written in any of a number of high level languages such as FORTRAN, PASCAL, JAVA, C, C++, C#, BASIC, various scripting languages, and/or HTML. Additionally, the software can be implemented in an assembly language directed to the microprocessor resident on a target computer. The software may be embodied on an article of manufacture including, but not limited to, a floppy disk, a jump drive, a hard disk, an optical disk, a magnetic tape, a PROM, an EPROM, EEPROM, programmable gate array, or CD-ROM, Embodiments using hardware circuitry may be implemented using, for example, one or more FPGA, CPLD or ASIC processors.

Alternatively, referring to FIG. 1B, the transducer may be a phased-array ultrasound transducer 106 formed by multiple transducer elements 130 made of piezoelectric materials. The transducer 106 may have an overall concave or bowl shape and the transducer elements 130 may be concentric rings. Typically, each of the rings will have substantially the same surface area, and thus the widths of the rings are progressively smaller from the innermost ring outward to the outermost ring. Alternatively, the rings may have equal widths, such that the area of each ring is progressively larger from the innermost ring to the outermost ring. Any spaces (not shown) between the rings may be filled with silicone rubber or the like to substantially isolate the rings from one another. In some embodiments, each ring is divided circumferentially into multiple curved elements or “sectors” that can be individually driven by the driver circuitry 124. It should be stressed that these known geometries are exemplary only. The focusing principle of a phased-array transducer is achieved by constructive interference due to the planned phase differences of the waves emitted by the individual elements, and any geometry suitable to this mode of operation (including even a completely flat transducer with a cartesian array of square elements) may be used to advantage.

Alternatively, a “composite” transducer with piezoceramic rods distributed within a polymer matrix can form the elements. Drive signals 132 generated by the driver circuitry 124 may be controlled by the controller 128. For example, the controller 128 may control the amplitude of the drive signals 132 to dictate the energy of the acoustic field delivered by the transducer 106. In addition, the controller 128 may control the relative phases and amplitudes of the signals driving the transducer elements 130. By shifting the phases between the transducer elements 130, a focal distance (i.e., the distance from the transducer 106 to the center of the focal zone 134), and the size, shape, and lateral position of the focal zone 134 may be adjusted. By changing the relative phase settings over time, the phased-array transducer 106 can be used to provide a two- or three-dimensional scan and, thus, obtain more detailed information about the target at the focal zone.

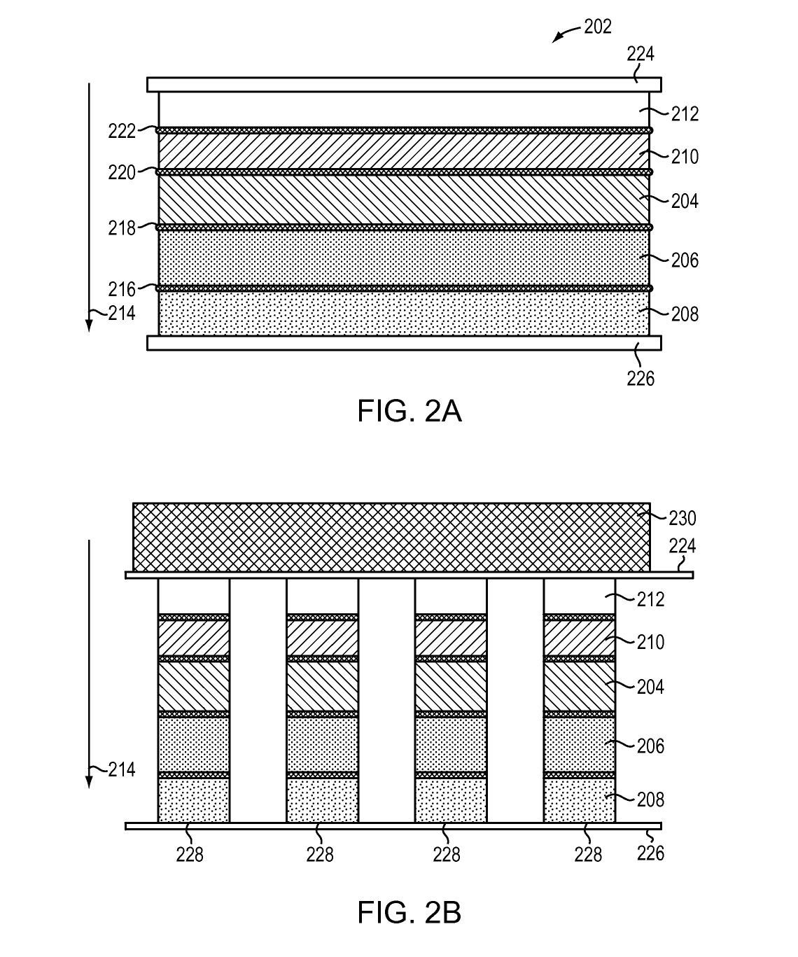

Referring to FIG. 2A, the single-plate transducer 104 and/or composite phase-array transducer 106 may include a multilayer structure 202 laminated in a stacked configuration; the number and order of the multiple layers and the material and thickness of each layer may be determined based on the desired power output and/or frequency response profile. For example, the multilayer transducer 202 may be configured to transmit acoustic waves at a transmission frequency and receive multiple harmonics of the transmission frequency with broadband sensitivity; this provides various advantages in ultrasound applications. For example, because the harmonic frequencies have higher signal-to-noise ratios than the transmission frequency, the use of the harmonic frequencies can enhance the resolution of ultrasound imaging. Additionally, the detection and processing of harmonic scatter may be used to characterize the static and dynamic properties of the transmission medium (e.g., tissue). Further, because temperature changes may significantly affect the propagation of the acoustic wave, the resultant attenuation of the detected harmonics in the received echoes can be used to derive temperature sensitive properties and/or the temperatures of the tissue. Accordingly, in various embodiments, the transducer 202 can be used to monitor therapeutic heating technologies, such as high intensity focused ultrasound (“HIFU”), ultrasound-induced hyperthermia, and/or other ultrasound and non-ultrasound based treatments. For example, while the first transducer 202 performs ultrasound treatment, the second transducer 202 may monitor the temperature changes of the target. Alternatively, the transducer 202 itself may perform both ultrasound treatment and temperature monitoring of the target.

In various embodiments, the multilayer transducer 202 includes a piezoelectric layer 204 that can be driven by electric signals to produce ultrasound energy. The piezoelectric layer 204 can be made of a variety of materials, such as ceramic, single crystal, polymer and co-polymer material, and/or ceramic-polymer material so that, typically, the layer 204 can resonate at a frequency between 20 kHz and 20 MHz for diagnostic and/or treatment purposes. In addition, materials in the piezoelectric layer 204 preferably have high electro-acoustic conversion efficiencies for transmitting high-power acoustic waves. In some embodiments, some or all other layers 206-212 of the transducer 202 are formed of conductive materials (e.g., having non-zero electrical conductivity or having volume resistivity less than 5 Ω×m) for efficiently delivering the output power. Such conductive materials include metals, graphite, carbon, plastics, conductive fiber composites, etc. Alternatively, non-conductive materials may be used in one or more (e.g., all) of layers 206-212. For example, non-conductive materials may be converted to z-conductive (i.e., conductive in the direction of the acoustic axis 214) materials by using holes and vias that are widely utilized in printed circuit board (PCB) technology. Additionally, one or more of the layers 206-212 may be coated with conductive materials (e.g., metals) to improve electrical performance.

The materials and thicknesses of the layers 206-212 may be chosen based on the acoustic and/or electromechanical properties thereof, the desired frequency response of the transducer, and/or the location of the imaging/treating target. For example, one or more of the layers 206-212 may include materials having different sound velocities, densities and attenuations. For example, a low density material can be sandwiched by high density materials producing a resonator at specific frequency. Different resonators for different frequencies within the same structure may be suitable for imaging/treating the target at different depths in the tissue, and may cover a large treatment area extending over 1-20 cm, for example). The piezoelectric layer 204 and/or other layers 206-212 may include materials such as piezopolymers or copolymers that have a wide bandwidth for receiving reflected acoustic waves with a large range of frequencies from the target. For example, depending on the acoustic properties of the stacked layers 204-212, the transducer 202 may detect up to the fifth harmonic (or higher harmonics) of the transmission frequency.

In various embodiments, the transducer 202 includes multiple piezoelectric layers; each layer has a different polarization direction for allowing different modes of vibration. Additionally, the transducer 202 may further include a dielectric layer (not shown) made of materials having high dielectric permeability (e.g., typically higher than 1000 relative permeability) for providing enhanced polarizability and thereby increasing power-delivery efficiency. Examples of the materials having high dielectric permeability include ceramics used in ceramic capacitor applications and depoled piezo-ceramics. In some embodiments, the dielectric layer is directly deposited onto the piezoelectric layer 204 by conventional thin film techniques, such as spin coating, dip coating or photolithography.

Referring again to FIG. 2A, in some embodiments, a series of interlayers 216-222 intervene between (and connect) adjacent layers 204-212 of the transducer 202. The interlayers 216-222 may be at least z-conductive (i.e., conductive in the direction of acoustic axis 214). For example, the interlayers 216-22 may be adhesives filled with conductive particles of any type (e.g., metal, graphite, carbon, metal-coated polymer, glass, or ceramic) that can ensure conductivity and lamination between the layers 204-212 when the transducer 202 is manufactured under high pressure. Other conventional interfacial adhesion layers that are well known in the semiconductor and/or microfabrication fields may also be applied between the layers 204-212. In some embodiments, the interlayers 216-222 are patterned with holes and vias also on the layers 204-212; the latter are made of non-conductive materials such that the entire stack 202 is z-conductive due to the holes, vias, and interlayers 216-222. Additionally, a surface-roughing treatment may be applied to one or more layers 204-212 of the transducer 202. In this case, electrical contacts between any two layers 204-212 may occur at the protrusion points of the rough surfaces; thus, the interlayers 216-22 may include a non-conductive adhesive material for filling the space between the electrical contacts and facilitating layer lamination during transducer manufacture.

Upon lamination of the multiple layers 204-212, the entire stack may be coated with a ground electrode layer 224 and a signal electrode layer 226 on the back and front (defined by the transmitting direction of the acoustic waves or acoustic axis), respectively, of the stacked transducer 202 using, for example, a physical deposition technique (e.g., evaporation or sputtering) that can provide a conformal coating to the surfaces of the stack. Accordingly, the electrode layers 224, 226 are oriented substantially parallel to the layers 204-212 and normal to an acoustic axis 2114, facilitating application of a voltage across the multilayer stack to generate acoustic energy. The electrode layers 224, 226 may include any metalized materials having low resistivity at a frequency between 100 kHz to 100 MHz, as would be understood by one skilled in the art.

The entire stack of the multilayer transducer 202 including the electrodes 224, 226 may then function as a single-plate transducer 204 or a composite phase-array transducer having multiple transducer elements 228 that are formed by segmenting the transducer stack using standard techniques (e.g., laser cutting or dicing) as shown in FIGS. 2A and 2B, respectively. In one implementation, no functional layers are required outside the two electrode layers 224, 226. Functional layers, such as layers 204-212 and interlayers 216-222, contribute to ultrasound energy transmission and reception.

In another embodiment, an impedance-matching layer 230 having a predetermined acoustic and/or electrical impedance and target thickness is utilized to passively improve electro-acoustic conversion efficiency of the stacked transducer 202 and/or maximize power delivery in the forward direction (i.e., in the direction of the arrow on the acoustic axis 214). Referring to FIG. 2B, the impedance-matching layer 230 may be positioned behind the ground electrode layer 204 to provide support thereto. Additionally, the impedance-matching layer 230 may increase the conversion efficiency of acoustic energy to electrical energy during reception. During manufacture, the matching layer 230 may be first applied to the surface of the electrode layer 204, allowed to cure and then lapped to the correct target thickness. The specific thickness range of the matching layer 230 depends on the actual choice of layer, its specific material properties, and/or the intended working center frequency of the transducer.

As described above, the multilayer transducer 202 in the current invention can provide a desired frequency response profile through the choice and order of the layers 204-212 and materials and thickness of each layer in accordance with its acoustic and electromechanical properties. Because the layers 204-212 are joined together (e.g., laminated) in a flat stack, the transducer will be homogeneous and thereby provide high-efficiency power delivery. In addition, the conductive materials and/or ceramics utilized in the multilayer transducer 202 have high thermal conductivities this ensures effective cooling of the transducer during operation. Further, virtually any conventional transducer construction technology that uses flat piezoelectric layers as a material for generating acoustic energy may be readily adapted to manufacture the multilayer transducer 202.

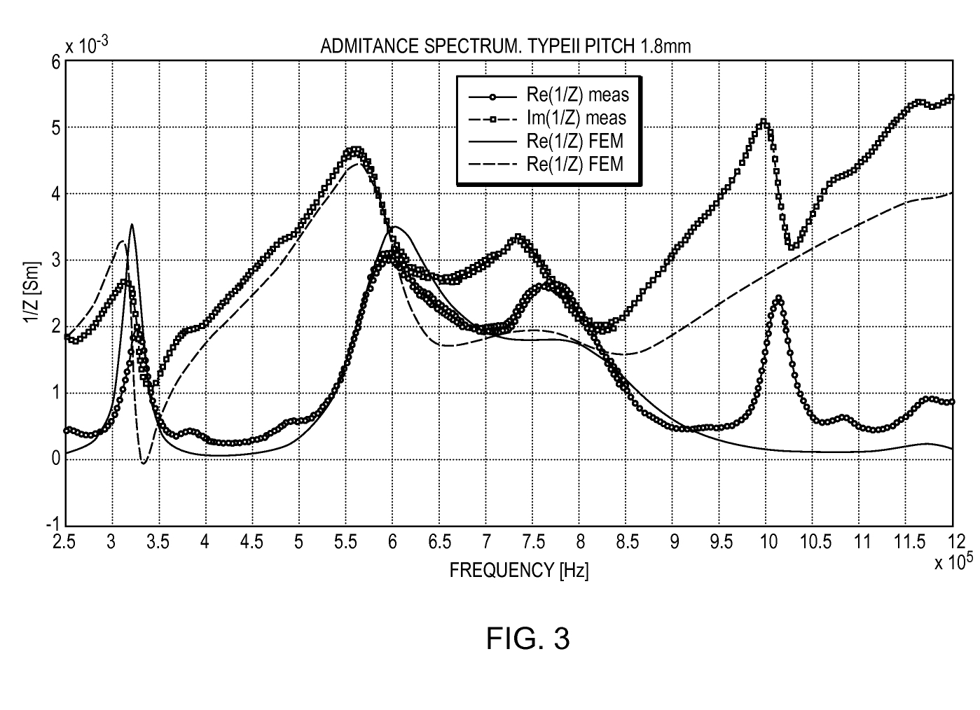

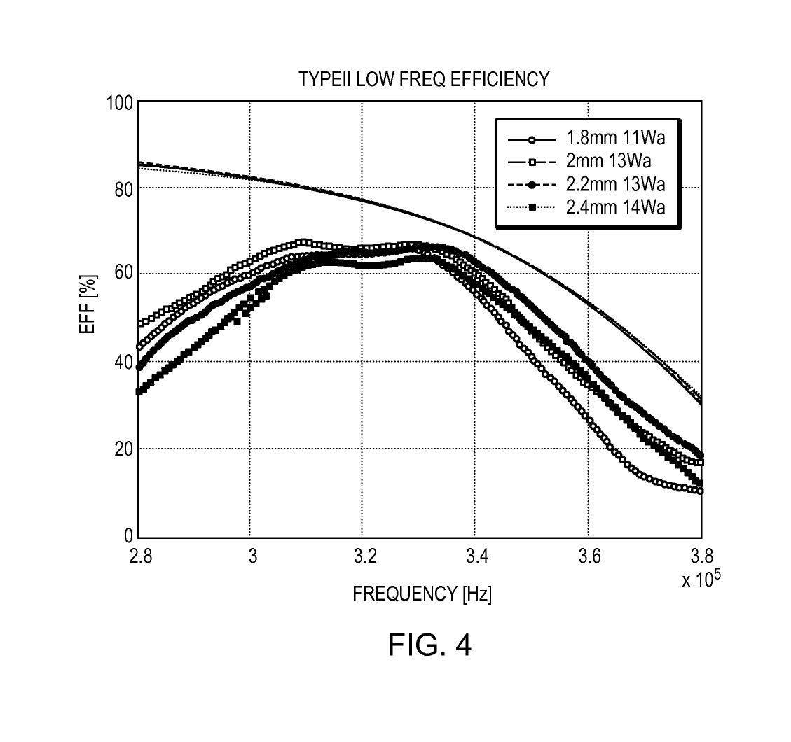

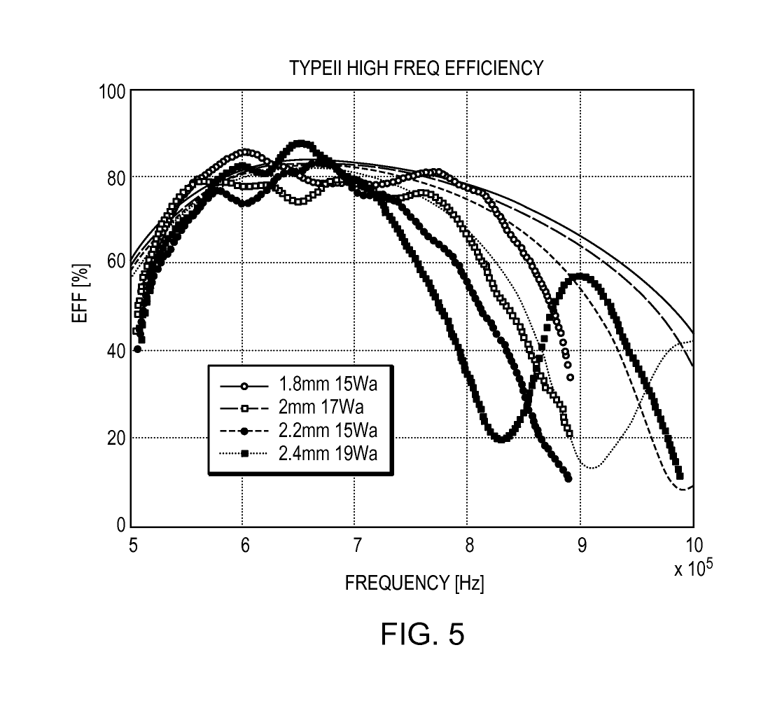

To design the multilayer transducer 202, the entire stack may be first regarded as a standing-wave resonator having a length equal to an integer number of half wavelengths (e.g., n×half-wavelength, where n is an integer). Therefore, the time-of-flight τ through the entire stack may be given as:

$$τ = \frac{n}{2 f} = {\sum }_{i} \frac{{d}_{i}}{{c}_{i}}$$

where f is the desired working frequency, and dᵢ and cᵢ are thickness and sound velocity, respectively, of the ith layer. In some embodiments, the piezoelectric layer 204 is assumed to be the standing-wave node. Based on these principles, a numerical simulation (e.g., finite element simulation) can be used to determine the materials and thickness of each layer. FIG. 3 depicts an admittance spectrum of a three-layered transducer whose materials are determined based on the simulation; the transducer includes a layer of resin-impregnated graphite (facing water), a layer of lead zirconate titantate (PZT), and a layer of copper-impregnated graphite (facing air) without electrical impedance matching. The three-layered transducer is segmented into multi-elements to create a composite transducer. The illustrated three-layered transducer generates ultrasound waves having dual frequencies at approximately 320 kHz and 550 kHz; the bandwidths at 320 kHz and 550 kHz are roughly 50 kHz and 330 kHz, respectively. The simulated results generally match the measurements. FIGS. 4 and 5 depict the efficiencies of the three-layered transducer simulated/measured at the low frequency (˜320 kHz) and high frequency (˜550 kHz), respectively—i.e., 60% at the low frequency and more than 80% at the high frequency. Accordingly, the three-layered transducer may be suitable for ultrasound applications involving working frequencies at approximately 320 kHz and 550 kHz with a bandwidth of 330 kHz at the high working frequency (550 kHz). The current invention thus provides an approach for creating a multilayer transducer; in the first step, a simulation may be used to determine various parameters (e.g., numbers and order of the layers, and materials and thickness of each layer) of the transducer using the approach described above, and in the second step, measurements of the actual performance of the designed transducer may be used to finely adjust the parameters of the transducer for achieving a desired frequency response and power delivery efficiency.

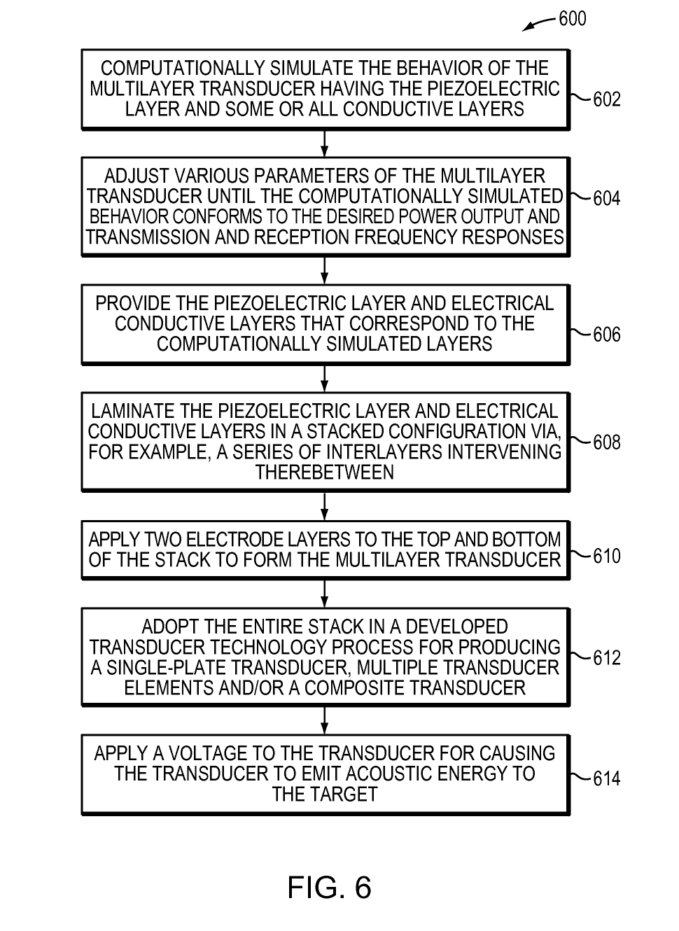

A representative method 600 illustrating the approach of design, manufacture, and use of the multilayer transducer in accordance with a desired power output and transmission and reception frequency responses is shown in FIG. 6. In a first step 602, a computational simulation is used to simulate the behavior of the multilayer transducer having the piezoelectric layer 204 and some or all conductive layers 206-212. In a second step 604, various parameters (e.g., the number and order of the layers 204-212, and materials and thickness of each layer) of the multilayer transducer are adjusted based on the intrinsic electrical and acoustic properties of the layers until the computationally simulated behavior conforms to the desired power output and transmission and reception frequency responses. The transducer may then be produced by first providing the piezoelectric layer 204 and electrical conductive layers 206-212 that correspond to the computationally simulated layers (step 606). The piezoelectric layer 204 and the electrical conductive layers 206-212 may then be laminated in a stacked configuration via, for example, a series of interlayers 216-222 intervening therebetween (step 608). Two electrode layers may be applied to the top and bottom of the stack to form the multilayer transducer (step 610). The entire stack can be adopted in a developed transducer technology process for producing a single piezomaterial plate (or a single-plate transducer), multiple transducer elements or/and a composite transducer (step 612). During operation (e.g., ultrasound imaging/therapy), a voltage is applied to the transducer for causing the transducer to emit acoustic energy to the target (step 614).

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Although the present invention has been described with reference to specific details, it is not intended that such details should be regarded as limitations upon the scope of the invention, except as and to the extent that they are included in the accompanying claims. Accordingly, the described embodiments are to be considered in all respects as only illustrative and not restrictive.

What is claimed is:

1. A transducer for delivering acoustic energy to a target site within a patient, the transducer comprising:
at least one piezoelectric layer;
a plurality of electrically conductive layers; and
two electrode layers;
wherein the at least one piezoelectric layer and the plurality of electrically conductive layers are positioned between the two electrode layers to form a stacked configuration that provides a desired power output and transmission and reception frequency responses.

2. The transducer of claim 1, wherein the at least one piezoelectric layer comprises at least one of ceramic, single crystal, polymer and co-polymer material, or ceramic-polymer.

3. The transducer of claim 1, wherein the electrically conductive layers have a volume resistivity of less than 5MΩ×m/F.

4. The transducer of claim 1, wherein the electrically conductive layers comprise at least one of metal, graphite, carbon, plastic, or conductive fiber composite.

5. The transducer of claim 1, wherein the transducer further comprises at least one interlayer connecting the at least one piezoelectric layer and the electrically conductive layers.

6. The transducer of claim 5, wherein the at least one interlayer comprise at least one of metal, graphite, carbon, metal-coated polymer, glass, or ceramic for ensuring conductivity and lamination between the at least one piezoelectric layer and the electrically conductive layers.

7. The transducer of claim 1, wherein the transducer further comprises a dielectric layer stacked between the two electrode layers.

8. The transducer of claim 7, wherein the dielectric layer comprises a ceramic or a depoled piezo-ceramic.

9. The transducer of claim 1, wherein the transducer further comprises an impedance-matching layer having a predetermined acoustic and/or electrical impedance and thickness.

10. The transducer of claim 1, wherein the transducer comprises no functional layers outside the two electrode layers.

11. A method of manufacturing and using a transducer, the method comprising:
providing a single piezoelectric layer and a plurality of electrically conductive layers;
laminating the single piezoelectric layer and the plurality of electrically conductive layers in a stacked configuration;
applying one electrode layer on top of the stack and one electrode layer on bottom of the stack to form a transducer; and
applying a voltage to the transducer, the voltage causing the transducer to emit acoustic energy,
wherein at least one of a material, thickness, or order of the layers is determined based on a desired power output and transmission and reception frequency responses.

12. The method of claim 11, wherein the electrode layers are added to the stack using evaporation or sputtering that provides a conformal coating to surfaces of the stack.

13. The method of claim 11, further comprising providing a plurality of interlayers connecting the single piezoelectric layer and the electrically conductive layers.

14. The method of claim 11, further comprising providing a dielectric layer stacked between the two electrode layers.

15. The method of claim 11, further comprising providing an impedance-matching layer having a predetermined acoustic and/or electrical impedance and thickness.

16. The method of claim 15, wherein the acoustic and/or electrical impedance of the impedance-matching layer is determined based on acoustic and/or electrical properties of the transducer.

17. The method of claim 11, further comprising segmenting the transducer into multiple elements for creating a composite phase-array transducer.

18. The method of claim 17, wherein the transducer is segmented using laser cutting or dicing.

19. A method of designing and manufacturing a transducer based on a desired power output and transmission and reception frequency responses, the method comprising:
computationally simulating behavior of at least one piezoelectric layer and least one electrical conductive layer;
adjusting at least one of (a) a number of layers, (b) an order of layers and (c) a thickness of the layers until the computationally simulated behavior conforms to the desired power output and transmission and reception frequency responses; and
producing the computationally simulated transducer by:
providing at least one piezoelectric layer and at least one electrical conductive layer corresponding to the computationally simulated layers;
laminating the at least one piezoelectric layer and the at least one electrical conductive layer in a stacked configuration; and
applying one electrode layer on top of the stack and one electrode layer on bottom of the stack.

20. A system for delivering acoustic energy to a target site within a patient, the system comprising:
a transducer having a plurality of layers comprising at least one piezoelectric layer, a plurality of electrically conductive layers, and two electrode layers configured in a stacked configuration;
driver circuitry for providing electrically drive signals to the transducer; and
a controller coupled to the driver circuitry for controlling the drive signals.
